(19) United States
(12) Patent Application Publication
(10) Pub. No.: US 2026/0102572 A1
(43) Pub. Date: Apr. 16, 2026

(54) AEROSOL GENERATOR FOR NEBULIZER

(71) Applicant: VADI Medical Technology Co., Ltd., Taoyuan City (TW)

(72) Inventor: Wei-Shin HUANG, Taoyuan City (TW)

(21) Appl. No.: 19/018,185

(22) Filed: Jan. 13, 2025

(30) Foreign Application Priority Data
Oct. 14, 2024 (TW) 113211054

(51) Int. Cl. A61M 11/00

(52) U.S. Cl. CPC A61M 11/006; A61M 2205/0211; A61M 2205/0233

(57) ABSTRACT

An improved aerosol generator for nebulizer comprises a vibrating aperture plate, a ceramic conducting strip, an electrical connector, and a contact pin head, the vibrating aperture plate being attached above the ceramic conducting strip, the fixed end of the electrical connector being welded to the periphery of the ceramic conducting strip, the contact pin head contacting the free end of the electrical connector to transmit electric power; wherein flexible flat cables are configured between the fixed end of the electrical connector and the free end of the electrical connector, so that the vibration caused by actuation of the ceramic conducting strip and the vibrating aperture plate can be absorbed by the flexible flat cables, and will not be transmitted to the free end of the electrical connector and the contact pin head.

### CROSS-REFERENCE TO RELATED APPLICATION

The present application claims priority under 35 USC 119 to Taiwan utility patent application No. 113211054, filed Oct., 14, 2024, which is hereby incorporated by reference in its entirety.

### BACKGROUND

### Technical Field

The present invention relates to a nebulizer, and more particularly to an improved aerosol generator.

### Description of Related Arts

A nebulizer utilizes vibrations to aerosolize medicinal liquid into aerosol, which is inhaled into the respiratory passages to cure bronchiectasis, diminish inflammation, or reduce phlegm. The aerosol generator is the actuating mechanism to aerosolize medicinal liquid.

US 20230001107 A1 discloses an aerosol generator, which at least comprises a vibrating aperture plate, an annular support to support the vibrating aperture plate, and a contact pin head pressed on the vibrating aperture plate to transmit electric power to the vibrating aperture plate.

U.S. Pat. No. 10,322,432 B2 discloses a multipoint contact pin head to reduce fretting wear resulting from long-term vibration by increasing multipoint contact between the contact pin head and the vibrating aperture plate.

### BRIEF SUMMARY OF THE INVENTION

The inventor is a professional manufacturer of medical devices and has rich experience in this industry. In view of the problem of reduced durability due to fretting wear of the contact pin head pressed on the vibrating aperture plate to supply electric power to the vibrating aperture plate after long-term vibration and the elastic fatigue resulting from long-term vibration of the spring inside the contact pin head. An improved aerosol generator for nebulizer is provided to effectively solve the above-mentioned problem.

Therefore, the present invention provides an improved aerosol generator for nebulizer, comprising a vibrating aperture plate, a ceramic conducting strip, an electrical connector, and a contact pin head, the vibrating aperture plate being attached above the ceramic conducting strip, the fixed end of the electrical connector being welded to the periphery of the ceramic conducting strip, the contact pin head contacting the free end of the electrical connector to transmit electric power; wherein flexible flat cables (FFC) are configured between the fixed end of the electrical connector and the free end of the electrical connector, so that the vibration caused by the actuation of the ceramic conducting strip and the vibrating aperture plate can be absorbed by the flexible flat cables, and will not be transmitted to the free end of the electrical connector and the contact pin head.

Other objects, features, and advantages of the present application will become apparent from the following detailed description, the accompanying drawings, and the appended claims.

### BRIEF DESCRIPTION OF THE DRAWINGS

FIG. 1 is a structure diagram of the nebulizer;

FIG. 2 is a exploded view of the aerosol generator; and

FIG. 3 is an assembly view of the aerosol generator.

### DETAILED DESCRIPTION OF THE INVENTION

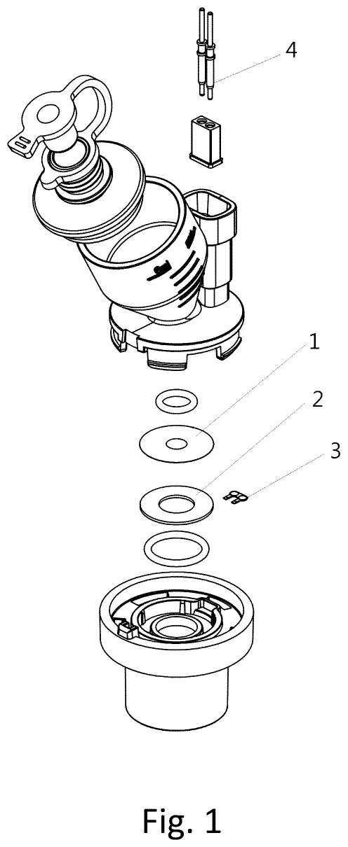

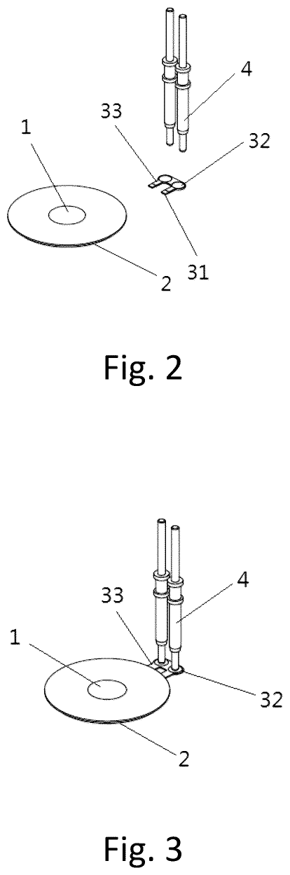

FIGS. 1-3 show an improved aerosol generator for nebulizer, comprising a vibrating aperture plate 1, a ceramic conducting strip 2, an electrical connector 3, and a contact pin head 4, the vibrating aperture plate 1 being attached above the ceramic conducting strip 2, the fixed end of the electrical connector 31 being welded to the periphery of the ceramic conducting strip 2, the contact pin head 4 contacting the free end of the electrical connector 32 to transmit electric power.

The improvement of the present invention lies in the flexible flat cables 33 configured between the fixed end of the electrical connector 31 and the free end of the electrical connector 32. Through this configuration, the vibration caused by actuation of the ceramic conducting strip 2 and the vibrating aperture plate 1 can be absorbed by the flexible flat cables 33, and will not be transmitted to the free end of the electrical connector 32 and the contact pin head 4.

In summary, the feature of the present invention lies in an electrical connector with flexible flat cables which apparently absorb the vibration caused by actuation of the ceramic conducting strip and the vibrating aperture plate. Therefore, the free end of the electrical connector and the contact pin head can be maintained at a constantly contacting state without vibration which effectively solves the problem of reduced durability due to fretting wear of the contact pin head and elastic fatigue of the spring inside the contact pin head due to long-term vibration.

Although the present patent application has been described in detail for the purpose of illustration, it is to be understood that such detail is solely for that purpose and that the present patent application is not limited to the disclosed embodiment, but, on the contrary, is intended to cover modifications and equivalent arrangements that are within the spirit and scope of the appended claims.

What is claimed is:

1. an improved aerosol generator for nebulizer, comprising:
a vibrating aperture plate,
a ceramic conducting strip,
an electrical connector, and
a contact pin head,
the vibrating aperture plate being attached above the ceramic conducting strip, the fixed end of the electrical connector being welded to the periphery of the ceramic conducting strip, the contact pin head contacting the free end of the electrical connector to transmit electric power;
wherein flexible flat cables are configured between the fixed end of the electrical connector and the free end of the electrical connector, so that the vibration caused by actuation of the ceramic conducting strip and the vibrating aperture plate can be absorbed by the flexible flat cables, and will not be transmitted to the free end of the electrical connector and the contact pin head.
